(19) United States
(12) Patent Application Publication
(10) Pub. No.: US 2010/0256420 A1
(43) Pub. Date: Oct. 7, 2010

(54) EXTRACTION AND PURIFICATION OF FRIEDELIN

(75) Inventors: Ricardo Alexandre Rodrigues Pires, Lisboa (PT); Susana Pinto Araújo da Silva Estima Martins, Maia (PT); José António Marchão das Chagas, Silvade (PT); Rui Luis Gonçalves dos Reis, Porto (PT)

Correspondence Address: MH2 TECHNOLOGY LAW GROUP, LLP, 1951 KIDWELL DRIVE, SUITE 550, TYSONS CORNER, VA, 22182, US

(73) Assignee: Amorim Revestimentos, S.A., S, Paio De Olerios (PT)

(21) Appl. No.: 12/746,356

(22) PCT Filed: Dec. 5, 2008
(86) PCT No.: PCT/PT08/00053

§ 371 Date: Jun. 4, 2010

(30) Foreign Application Priority Data
Dec. 6, 2007 (EP) 07398018.7

(51) Int. Cl. C07C 45/81; C07C 45/83

(52) U.S. Cl. 568/366

(57) ABSTRACT

Friedelin is a natural compound with promising proprieties. On its own or with chemical modification it is possible to introduce relevant biological activities, e.g., anti-cancer, anti-aging and agrochemical. Its availability in significant amounts has been a major drawback on its regular use and in the pursuit of different applications. Cork and cork-derived materials (e.g., black condensate) are the most relevant sources of Friedelin in nature (up to 10% in concentration). The present invention reports straight-forward procedures to extract and purify Friedelin from cork and cork-derived materials (e.g., black condensate). It uses solvent extraction and (re)crystallization techniques easy to scale up to an industrial level, with a low solvent and low time consumption, high yields, up to 2.9%, and high purity degrees, up to 96%.

### FIELD OF THE INVENTION

Friedelin is one of the most abundant terpenes present in cork and cork-derived materials. Recent studies showed that Friedelin and, particularly, its derivates have anti-cancer activity (Moiteiro et al., J. Nat. Prod., 64 (2001), 1273-1277; Moiteiro et al., J. Nat. Prod., 67 (2004), 1193-1196; Moiteiro et al., WO09026, 1994; Zhang et al., WOQ52383A1, 2004), analgesia and anti-inflammatory capability (Nakamura et al., EP0776666A2, 1997), anti-bacterial activity (Frame, A. D., WQ059371A1, 2003), vascularizing agent (Mitsuhiro et al., JP068972, 2002), have potential to be used in pharmaceuticals or functional foods for the treatment or prevention of cardiovascular and cerebrovascular diseases and tumours (Zhang et al., U.S. Pat. No. 0,148,733, 2006), potential to be used in cosmetics (Kinshi et al., JP012509, 2003) and as agrochemical (Moiteiro et al., J. Agric. Food Chem., 54 (2006), 3566-3571). Its anti-oxidizing capacity is under study.

The availability of Friedelin in large quantities has been a major drawback in the development of applications with industrial relevance. The procedures described in this invention allow the extraction of Friedelin from cork and cork-derived materials, such as cork extracts, cork powder, granules, planks, but in particular from one of the cork industry by-products: black condensate.

Black condensate is usually produced in large quantities by the insulation corkboard industry. This by-product has no relevant application and is usually incinerated. This invention gives an added value to this by-product as a source of fine and speciality chemicals (e.g., Friedelin).

### SUMMARY OF THE INVENTION

The present invention describes a straightforward procedure of extraction and purification of Friedelin from cork and cork-derived materials by means of alkaline hydrolysis, solvent extraction/washing and (re)crystallization techniques using low amounts of isolated or mixtures of solvents. With the present invention, it is possible to obtain Friedelin with a purity degree of up to 96% and with an overall extraction yields of up to 2.9% (corresponding to, approximately, 30% to 50% of the amount of Friedelin present in the raw material)

The present invention relates to a process for Friedelin extraction from cork or cork-derived materials characterized by comprising the following steps of:
(i) washing of said materials with an aliphatic alcohol or a solution thereof, for time periods up to 24 h and from room temperature to the boiling temperature of the solvent;
(ii) extraction of washed raw material with an apolar solvent, for time periods up to 24 h and from room to reflux temperatures, and evaporation of the solvent thus yielding a so-called Friedelin-rich solid extract;
(iii) purification of the Friedelin-rich extract by (re)crystallization using halogenated solvents, aliphatic hydrocarbons, aliphatic alcohols, ketones, ethers, esters, carboxylic acids and/or acetates as isolated solvents or mixtures, thus yielding a so-called Friedelin-rich material;
optionally followed by:
(iv) solubilisation of the Friedelin-rich material in one of the solvents used in step (iii) with increased temperature and with the addition of activated charcoal, followed by filtration and evaporation of the solvent.

In one aspect of the invention, said cork-derived materials are black condensate.

In another aspect of the invention, the aliphatic alcohol solution of step (i) comprises an alkali, which is selected from calcium carbonate, magnesium hydroxide, potassium or sodium hydroxide.

In another aspect, the washing step (i) is performed at the boiling temperature during 6 hours.

In still another aspect of the present invention, an additional washing step of the filtration residue with an aliphatic alcohol is performed between steps (i) and (ii) in order to improve the purity of the Friedelin-rich solid extract.

In another aspect, the apolar solvent of step (ii) is selected form the group comprising chloroform, methylene chloride and diethyl ether.

In still another aspect of the present invention, the purification step (iii) is performed in the presence of activated charcoal.

In another aspect of the invention, the (re)crystallization of step (iii) is repeated in order to obtain higher purity samples.

In still another aspect, an additional washing step with an aliphatic alcohol is performed after step (iv) in order to separate contaminant remains.

In still another aspect, the aliphatic alcohol used in previous aspects of the invention is methanol, ethanol, 1-propanol, 1-butanol, 2-butanol or isopropanol.

### BACKGROUND OF THE INVENTION

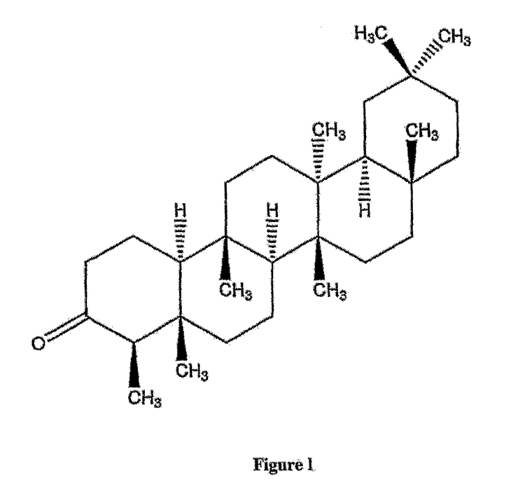

Cork is one of the most relevant sources of Friedelin in nature. Natural and treated cork, in powder or granules, is described to have a significant amount of Friedelin (chemical structure presented in FIG. 1). Additionally, during insulation corkboard production a by-product called black condensate is formed. Having no relevant application is usually incinerated, although, it is a Friedelin-rich material (1-10% in weight concentration) (Sousa et al., J. Agric. Food Chem., 54 (2007), 6888-6893).

Procedures of isolating Friedelin from black condensate have been described (Júnior et al., PT101683A, 1996; Júnior et al., EP1103539A1, 2001) using organic solvent fractionation and extraction with aliphatic hydrocarbons, although, the described methodologies require multi-step procedures that include 5 to 11 steps to reach low purity Friedelin (40% to 60%) and 10 to 12 steps to obtain a sample of high purity Friedelin (≧95%). Additionally, considering the overall quantity of solvents used, the methodologies described in the literature consume between 0.92 L and 1.18 L of solvents per gram of high purity Friedelin (≧95%).

More recently, extraction and purification of terpenoids (including Friedelin) from Heritiera littoralis D. has been described using chromatographic fractionation technology (Tian et al., CN1580071, 2005). All the reported cases, describe procedures that are time and solvent consuming.

In the case of Friedelin and its derivatives, it has been reported several interesting biological activities (e.g., Moiteiro et al., WO09026, 1994). The anti-cancer potential of a Friedelin derivative (2,3-secofriedelan-2-al-3-oic acid) has been reported to have an inhibitory effect on the growth of breast, renal, melanoma, lung and central nervous system cancer cells (Moiteiro et al., J. Nat. Prod., 64 (2001), 1273-1277). Additionally, in the case of 3-nor-2,4-secofriedelan-4-oxo-2-oic acid (another Friedelin derivative) it was also reported its inhibitory action in the growth of breast, lung and central nervous system cancer cells (Moiteiro et al., J. Nat. Prod., 67 (2004), 1193-1196). More recently, Friedelin and its derivatives have been studied as agrochemicals for crop protection. The study showed promising results for several Friedelin derivatives against S. littoralis and L. decemlineata (Moiteiro et al., J. Agric. Food Chem., 54 (2006), 3566-3571).

The increasing number of possible applications for Friedelin and its derivatives demands for sources where it can be found in high concentrations. On the other hand, the development of Friedelin extraction, isolation and purification procedures with an industrially appealing yield, cost and purity, are needed. This invention pretends to give an answer to this demand, reporting a simple and straightforward solvent extraction procedure to obtain Friedelin from cork and cork-derived materials.

### DETAILED DESCRIPTION OF THE INVENTION

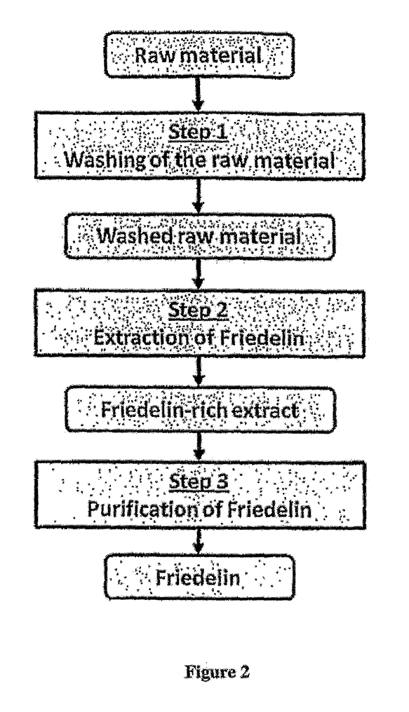

The present invention consists of a three to four steps procedure with a low time and low solvent consumption. The first step is to obtain a material with a reduced concentration of contaminants, from which is possible to extract Friedelin. The second step focuses on the extraction of Friedelin, while in the third step a purification of the extracted Friedelin is carried out. A scheme of the procedure is presented in FIG. 2.

At the first step a treatment (washing) of the raw material (e.g., black condensate) with an aliphatic alcohol for time periods up to 24 h and from room to the boiling temperature of the solvent is performed, preferably at the boiling temperature. The use of an alkaline alcoholic solution is preferred. At this step a Soxhlet experimental apparatus can also be used. Optionally, an additional washing of the filtration residue with an aliphatic alcohol can improve the purity of the final extract (obtained in the next step).

In the second step Friedelin is extracted from the washed raw material using solvent extraction techniques. In this step, an apolar solvent is preferably used (e.g., chloroform, methylene chloride, etc.), from room to reflux temperatures and from time periods up to 24 h. The evaporation of the solvent yields a Friedelin-rich solid extract.

The third step is the purification of the Friedelin-rich extract by (re)crystallization technique using halogenated solvents, aliphatic hydrocarbons, aliphatic alcohols, ketones, ethers, esters, carboxylic acids and/or acetates as isolated solvents or mixtures (e.g., n-hexane, n-hexane:ethyl acetate), with or without the addition of activated charcoal. This (re)crystallization step can be repeated to obtain higher purity samples.

An alternative or sequential technique (optional fourth step) is to solubilise the Friedelin-rich material in one of the previously described solvents (isolated or in mixtures) with increased temperature and with the addition of activated charcoal. Higher purity Friedelin can be obtained after filtration of the solution and evaporation of the solvent. If necessary, a final wash of the Friedelin sample with an aliphatic alcohol (e.g., ethanol) might be necessary to separate contaminant remains.

The described examples are based on black condensate due to its complex matrix and the higher probability of having contaminations during the purification steps. The described procedures can be executed with a lower consumption of solvent than previous patents. With the present straightforward four steps procedures it is possible to spend less than 0.55 L of solvent to obtain one gram of high purity Friedelin (≧95% purity). Additionally, taking the previously described into consideration, a successful procedure to extract Friedelin from black condensate can also be applied in less complex matrixes as, for example, most of the solvent extracts of cork and cork-derived materials.

### Example 1

Black condensate powder (m=204.9 g) was extracted with 2 L of 2% sodium hydroxide ethanolic solution during 6 hours under reflux. The mixture was cooled down to room temperature and filtrated under vacuum. The filtration residue was dried yielding 116.8 g of raw material for Friedelin extraction.

Chloroform (V=1170 mL) was added to the material obtained from the previous step (m=116.8 g) and stirred during 30 min at room temperature. The mixture was filtrated under vacuum and the solvent (that might be reused) was removed from the extract and collected using a rotator evaporator. The residual chloroform was evaporated at room temperature, yielding 18.5 g of a Friedelin-rich extract, corresponding to an extraction yield of 9.0%.

The Friedelin-rich extract (m=15.0 g) was dissolved in hot n-hexane:ethyl acetate system in the presence of activated charcoal. After filtration of the charcoal (re)crystallization yielded 8.1 g of Friedelin, corresponding to an overall extraction yield of 4.9%.

Higher purity Friedelin was obtained dissolving 1.0 g of Friedelin from the previous step in n-hexane hot and adding activated charcoal to the hot solution, under constant heating and stirring, for 30 min. The charcoal was filtered and the solvent was evaporated yielding 0.6 g of Friedelin.

The procedure described in this example produced Friedelin with a purity degree of 96% and an overall yield of extraction of 2.9%. This example is a four step procedure that uses 0.53 L of solvent per gram of high purity Friedelin.

### Example 2

Black condensate powder (m=204.3 g) was extracted with 2 L of 3% potassium hydroxide methanolic solution during 3 h under reflux. The mixture was cooled down to room temperature and filtered under vacuum. The filtration residue was dried yielding 148.7 g of raw material for Friedelin extraction.

Chloroform (V=1490 mL) was added to the material obtained from the previous step (m-148.7 g), stirred during 30 min at room temperature. The mixture was filtrated under vacuum and the solvent (that might be reused) was removed from the extract and collected using a rotator evaporator. The residual chloroform was evaporated at room temperature, yielding 11.8 g of a Friedelin-rich extract, corresponding to an extraction yield of 5.8%.

The Friedelin-rich extract (m=10.Og) was dissolved in hot n-hexane:ethyl acetate system in the presence of activated charcoal. After filtration of the charcoal (re)crystallization yielded 4.3 g of Friedelin, corresponding to an overall extraction yield of 2.5%.

The Friedelin obtained from the previous step (m=1.0 g) was dissolved in n-hexane hot. Activated charcoal was added to this solution, under constant heating and stirring, for 30 min. The charcoal was filtered and the solvent was evaporated yielding 0.5 g of Friedelin.

The procedure described in this example produced Friedelin with a purity degree of 61% and an overall yield of extraction of 1.2%.

1. A process for Friedelin extraction from black condensate comprising the following steps of:
(i) washing of said materials with an aliphatic alcohol or a solution thereof, for time periods up to 24 h and from room temperature to the boiling temperature of the solvent;
(ii) extraction of washed raw material with an apolar solvent selected from the group consisting of chloroform, methylene chloride and diethyl ether, for time periods up to 24 h and from room to reflux temperatures, and evaporation of the solvent thus yielding a Friedelin-rich solid extract;
(iii) purification of the Friedelin-rich extract by (re)crystallization using halogenated solvents, aliphatic hydrocarbons, aliphatic alcohols, ketones, ethers, esters, carboxylic acids and/or acetates as isolated solvents or mixtures, thus yielding a Friedelin-rich material;
optionally followed by:
(iv) solubilisation of the Friedelin-rich material in one of the solvents used in step (iii) with increased temperature and with the addition of activated charcoal, followed by filtration and evaporation of the solvent.

2. (canceled)

3. A process for Friedelin extraction according to claim 1, wherein the solution of step (i) is an alkaline aliphatic alcohol solution.

4. A process for Friedelin extraction according to claim 3, wherein the alkali of the alkaline aliphatic alcohol solution is calcium carbonate, magnesium hydroxide, potassium or sodium hydroxide.

5. A process for Friedelin extraction according to claim 1, wherein the washing step (i) is performed at the boiling temperature for up to 6 hours.

6. A process for Friedelin extraction according to claim 1, wherein an additional washing step of the filtration residue with an aliphatic alcohol is performed between steps (i) and (ii) in order to improve the purity of the Friedelin-rich solid extract.

7. (canceled)

8. A process for Friedelin extraction according to claim 1, wherein the purification step (iii) is performed in the presence of activated charcoal.

9. A process for Friedelin extraction according to claim 1, wherein the (re)crystallization of step (iii) is repeated in order to obtain higher purity samples.

10. A process for Friedelin extraction according to claim 1, wherein an additional washing step with an aliphatic alcohol is performed after step (iv) in order to separate contaminant remains.

11. A process for Friedelin extraction according to claim 1, wherein the aliphatic alcohol is methanol, ethanol, 1-propanol, 1-butanol, 2-butanol or isopropanol.

12. A process for Friedelin extraction according to claim 6, wherein the aliphatic alcohol is methanol, ethanol, 1-propanol, 1-butanol, 2-butanol or isopropanol.

13. A process for Friedelin extraction according to claim 10, wherein the aliphatic alcohol is methanol, ethanol, 1-propanol, 1-butanol, 2-butanol or isopropanol.
